(19) United States
(12) Patent Application Publication
(10) Pub. No.: US 2008/0248144 A1
(43) Pub. Date: Oct. 9, 2008

(54) Method of Producing a Cosmetic Abrasive

(75) Inventors: Daniel Guenter, Krefeld (DE); Michael Friebel, Toenisvorst (DE)

Correspondence Address: OBLON, SPIVAK, MCCLELLAND MAIER & NEUSTADT, P.C., 1940 DUKE STREET, ALEXANDRIA, VA, 22314, US

(73) Assignee: EVONIK STOCKHAUSEN GMBH,, KREFELD (DE)

(21) Appl. No.: 12/093,746

(22) PCT Filed: Nov. 13, 2006
(86) PCT No.: PCT/EP06/10845

§ 371 Date: May 15, 2008

(30) Foreign Application Priority Data
Nov. 16, 2005 (DE) 10 2005 054 976.4

(51) Int. Cl. A61K 8/97; C11D 3/382; A61Q 19/00; A61Q 19/10

(52) U.S. Cl. 424/776; 510/139; 424/777

(57) ABSTRACT

The invention relates to a method of producing an abrasive substance, where natural kernels, shells, fruit skins and/or seeds are ground to give a flour of defined particle size, the flour is treated in aqueous suspension with at least one bleaching agent, in which the addition of the bleaching agent takes place in two steps, where, in the first step, at least 40% by weight to 90% by weight of the total amount of bleaching agent is added and the flour is sterilized in the acidic medium and, in the second step, the lightening and degreasing of the flour is brought about by the simultaneous metered addition of the remainder of the bleaching agent with an alkali solution in alkaline medium, and its use in cosmetic preparations.

The present invention relates to a method of producing a cosmetic abrasive and to its use in cosmetic products.

An essential constituent of cosmetic cleansing and treatment compositions is the abrasive, which has the task of mechanically aiding the cleansing effect of washing-active or surfactant-like components.

The prior art describes numerous inorganic and organic materials which can be used in cleansing preparations as mechanical cleansing and treatment compositions, in particular in hand cleansers or in so-called peeling creams and special cleansing gels. They serve here to remove the upper dead skin cells or skin impurities, for example on the face or other parts of the body.

Of particular interest here are natural abrasives, such as, for example washed and ground shells of walnuts, and ground apricot kernels or olive kernels which, on account of their hardness and particle size, are suitable for superficial skin cleansing. Such natural abrasives have a gentle, very good cleansing effect without scratching the skin. A disadvantage of such natural abrasives is that they lead to cosmetic products which have a dark, dirty appearance. In order to prevent this, lightening pigments such as, for example, titanium dioxide, are added to these cosmetic products.

EP 0 559 696 B1 describes a method of producing material treated with a bleaching agent, in particular hydrogen peroxide, in fine distribution of natural shells and/or kernels, and illustrates the use of the thus obtained bleached abrasive in cosmetic products.

The method described in this patent specification for bleaching natural flours, which bleaches and dries flours of certain particle size, preferably walnut shell flour, has been used successfully on an industrial scale for many years. The flours produced by it usually have a germ count of less than 10² CFU/g and are free from pathogenic germs, have a pale beige color and are used in handwashing pastes for industrial hand cleansing. In this connection, the abrasive effect brought about by the flours has proven very adequate and effective for the physical removal of industrial soilings for many years.

Since the bleaching of the natural kernel and shell flours is carried out in hydrous suspensions, large amounts of waste liquors are also produced, as the extensive practice of this method has shown. Moreover, the method also uses stabilizers and reducing agents, which, being additional raw materials, naturally make this method more expensive compared with a possible method in which such stabilizers and reducing agents could be dispensed with.

To avoid such waste liquors, EP 1 136 063 A2 proposes a bleaching method in which, in a “dry” process, peracids are sprayed onto the natural flour, which can be obtained as biological material from a large number of plant materials, and lightening is said to be effected. The mixture of this biological material with the bleaching agent should comprise at most 60% by weight of water where, after the mixing operation, an after ripening process starts. After 10 days, the peroxide formed during the method should no longer be detectable in the resulting product.

However, a disadvantage of such a method is that residual amounts of carboxylic acids are present in the finished flour which, as a result of the formation of salt contents in the production of the cosmetic end products, especially in handwashing pastes, have an adverse environmental impact. Moreover, in this method, up to 5% by weight, based on the mixture comprising the biological material and the bleaching agent, of stabilizers in the form of moderators are used. The corresponding applies for the use of reducing agents for destroying excess peroxides.

DE 103 05 959 describes a method whose bleaching result with regard to germ content, odor and color leads not only to abrasives which, in terms of their profile of properties, are comparable with or better than the abrasives obtained by the method described in EP 0 559 696 B1 which, when used in cosmetic cleansing compositions with the lowest possible concentration of lightening substances, or without them entirely, produce optically light and cosmetically acceptable products. Furthermore, in this method, the use of stabilizers and reducing agents is also completely dispensed with in the production process. The wastewaters which form after the washing process of the microbiologically decontaminated, deodorized and bleached flours are improved with regard to their biodegradability and the overall production process also becomes more cost-effective from the point of view of operating costs.

In the aforementioned method, natural kernels, shells, fruit skins and/or seeds are ground to a flour of defined particle size. The resulting flour is then treated in aqueous suspension with 1.0 to 10.0% by weight of a bleaching agent, based on the total batch amount. Here, the addition of the bleaching agent takes place in two steps, where, in the first step, following the addition of from 20 to 40% by weight of the bleaching agent, based on the total amount of bleaching agent used, a pH range from 3 to 5 is obtained.

Nevertheless, in this two-stage process in the production operation, it has proven disadvantageous that the nature and amount of the bleaching agent metered addition, in particular of the hydrogen peroxide metered addition, could result, depending on the batch, in increased germ counts and very differentiated lightening:

The non-attainment of the required maximum germ count of 10² CFU/g in these batches—where in fact germ counts in the region of 10 CFU/g have been measured—resulted in containerized goods from big bags having to be constantly returned again to the bleaching process. For example, about 15% of the total amount of the flour to be bleached was returned to the process again as contaminated material.

This meant that the logistic steps to be carried out, such as warehousing, transportation and mixing processes were dependent on the result of the germ count determination and therefore had to be planned to take into account the unavoidable conveyance of these contaminated flours to a further bleaching process. The flours were converted to an adequate microbiological state by this second treatment so that they were then able to be conveyed to their use as intended as natural abrasives in cosmetic products.

The lightening process brought about by this method besides the sterilization likewise sometimes proved unstable in continuous production operation since now and then clearly visible differences in the end color of the treated flour resulted which was not only caused by the bandwidth of the starting color in the raw material. Thus, batches with a relatively dark color that cannot be tolerated for use in cosmetics also arose.

These additional labor-intensive processing steps, in particular the associated yield losses, ultimately lead to considerable additional costs for the overall production process.

Moreover, it has been recognized that, besides the sterilization and the lightness of the abrasives produced by the method, their fat content should also be taken into consideration since the fat content of the abrasives has an effect on the properties that should not be disregarded, in particular the viscosity of the cosmetics produced using the bleached abrasives, preferably skin and hand cleansing compositions such as, for example, coarse hand cleansers.

There thus continues to be a need for a production method for abrasives which are destined for cosmetic products which ensures an environmentally friendly and economic arrangement of the production process which leads to the microbiological decontamination, deodorization and bleaching and also degreasing of natural kernel and shell flours.

It was therefore an object to provide a method for bleaching natural kernels, shells, fruit skins and/or seeds for producing cosmetic abrasives which, even on an industrial or commercial scale, has a reject rate resulting from contaminations below 0.5% and a very good microbiological stability, where a uniform lightening/color is ensured at the lowest possible fat content over the entire bleaching campaign.

The above object was achieved according to the invention by a method of producing an abrasive substance, where natural kernels, shells, fruit skins and/or seeds are ground to give a flour of defined particle size, the flour is treated in aqueous suspension with at least one bleaching agent, where the addition of the bleaching agent takes place in two steps, where, in the first step, at least 40% by weight to 90% by weight of the total amount of bleaching agent are added and the flour is sterilized in the acidic medium and, in the second step, the lightening and degreasing of the flour is brought about by the simultaneous metered addition of the remainder of the bleaching agent with an alkali solution in alkaline medium.

According to the invention, the natural shell or kernel flours used are walnut shell flour, almond shell flour, hazelnut shell flour, olive kernel flour, apricot kernel flour, peach kernel flour, cherry kernel flour, plum kernel flour or other natural shell or kernel flour, for example from palm kernels and coconuts, jojoba fruits, macadamia nuts and other nuts, pistachios and pine shells and other kernel fruit, and also any desired mixture of said materials. According to the invention, a particularly preferred natural shell or kernel flour is walnut shell flour.

Moreover, further plant flours from fruit skins and seeds known as mild abrasives in the prior art, such as, for example, corn cob flour, wheat bran, oat flour, but also any desired wood flours, can also be bleached using the method according to the invention.

In order to obtain a defined particle size of the flours of natural kernels, shells, fruit skins and/or seeds to be used in the method according to the invention, these are ground in a manner known per se to give a flour, optionally with incorporation of a classification by sieve. Flours which have a particle size of from 50 to 2000 μm, preferably from 70 to 1000 μm and particularly preferably from 80 to 400 μm, can be used in the method according to the invention.

For grinding the flours, the comminution apparatuses or mills known in the prior art can be used, as have been detailed, for example, in EP 0 559 696, in particular impact mills with pendulum or plate impacter, passage roll mills, hammer impact or pin-type mills, optionally with classification units, such as, for example, Condux mills etc.

The natural kernels, shells, fruit skins and/or seeds ground to a flour of defined particle size are treated in aqueous suspension with at least one bleaching agent. Bleaching agents that can be used are all compounds which ensure irreversible destruction of the chromophores of these natural flours, the bleached flours being chemically unchanged, or only insignificantly chemically changed, during the bleaching treatment according to the invention so that they can be used as abrasives in cosmetic products. Such bleaching agents are, for example, so-called oxidizing bleaching agents, as are described, for example, in Ullmann's Encyclopedia of Industrial Chemistry, 4th edition, volume 8, pages 589 to 595. Preference is given to inorganic and organic peroxides, such as, for example, hydrogen peroxide, sodium peroxide, barium peroxide or peroxycarboxylic acids, in particular peroxyformic acid, peroxyacetic acid and peroxypropionic acid etc., which can be prepared in situ and used according to the invention in a known manner for the person skilled in the art. The aforementioned compounds can be used on their own or else as a mixture of at least two of these compounds in the method according to the invention. According to the invention, the bleaching agent is added to the bleach material in an amount of from 1.0% by weight to 10.0% by weight, preferably 1.0% by weight to 3.0% by weight, based on the total batch amount, where the preferred bleaching agent to be used is aqueous hydroxide peroxide solution.

The starting temperature for the treatment of the aqueous suspension of the flour of natural kernels, shells, fruit skins and/or seeds is 20 to 40° C., preferably 25 to 35° C. and particularly preferably 28 to 32° C.

It is essential for the present invention that the addition of the bleaching agent takes place in two steps, where, in the first step, the sterilization of the flour takes place in the acidic medium at the start of the bleaching process following the addition of at least 40 to 90% by weight of the bleaching agent, preferably 45 to 80% by weight, particularly preferably 50 to 70% by weight, based on the total amount of bleaching agent used. The pH of the medium here is in particular 3 to 5, preferably 4 to 5.

The subsequent addition of the remainder of the bleaching agent in the second stage for lightening and degreasing the bleach material takes place in the alkaline medium simultaneously with an alkali solution. In particular, 10 to 60% by weight, preferably 15 to 55% by weight, particularly preferably 30 to 50% by weight, of the residual amount of bleaching agent are metered in simultaneously with an alkali solution, where the lightening and degreasing of the bleach material is effected at a pH of from 7.0 to 11.0, preferably 7.3 to 9.0, particularly preferably 7.8 to 8.5.

The addition of the bleaching agent to the bleach material preferably takes place in liquid form in both steps for reasons of better metered addition and pH control. However, this addition form of the bleaching agent is not limited to this, but is dependent on the choice of bleach material and/or the bleaching agent to be used. Thus, it is also possible according to the invention to add the addition of the bleaching agent firstly in solid form to the bleach material suspension and, in the second step, to meter in the remainder of the bleaching agent in liquid form simultaneously with the alkali solution.

Since the method according to the invention is a large-scale industrial process, for reasons of economic feasibility, water is the particularly preferred solvent of the required bleaching agent. Nevertheless, it may also be appropriate here, in view of the type of bleach material and/or the required bleaching agent, to dissolve the bleaching agent in a customary organic solvent in order to achieve the desired bleaching effect. The resulting bleaching agent solution is then added, as described, in two steps to the suspension of the bleach material.

The alkali solutions used are usually aqueous solutions of hydroxides of the alkali metals, in particular of sodium and potassium hydroxide. Furthermore, it is also possible to use ammonium hydroxide and the hydroxides of the alkaline earth metals, and also the carbonates of the alkali metals, in particular sodium carbonate and potassium carbonate. However, particular preference is given to aqueous sodium hydroxide solution as alkali solution to be used according to the invention, in particular as 35 to 75% strength, preferably 45 to 60% strength and particularly preferably as 50% strength aqueous solution.

The process time of the method according to the invention is 160 to 320 minutes, preferably 180 to 220 minutes and particularly preferably 190 to 210 minutes.

On account of the gentle processing, the end temperature of the natural kernel, shell, fruit skin and/or seed flour suspension after the bleaching agent treatment, in particular with hydrogen peroxide solution, is merely about 15 to 20° C. above the starting temperature.

Water is then removed from the bleach suspension by customary known methods and the damp flour is washed with hot water from 70 to 95° C., preferably 80 to 93° C. and particularly preferably 85 to 92° C. and then subjected to an intensive drying process. The dried flour is containerized in large packs and passed directly to its use as abrasive in cosmetic preparations, for example in solvent-free or solvent-containing handwashing pastes, in anhydrous skin cleansing compositions and in peeling creams.

The advantages of the method according to the invention are specifically:

The process products obtained by the method according to the invention have not only a comparable, but sometimes even improved, product quality compared to the abrasives known in the prior art, especially with regard to the lightness of the products.

Thus, it was found, by means of physical color measurement using a calorimeter according to DIN 5033, that the process end products according to the invention have an L* value of at least 81.

The evaluation scale of the values which the L* value can assume ranges from 0 for ideally black colors to 100 for ideal white (see DIN 5033 and DIN standards cited therein; cf. also DIN 6174).

As a result of this, it was possible to render the color lightening more effective, which is advantageous for use in cosmetic products as regards the optical properties of the cosmetic end product.

The natural products bleached by the method according to the invention have, on an industrial or commercial scale, merely a reject rate as a result of contaminations below 0.5%, i.e. of at most 0.2%, as a result of which, compared to the method described in DE 103 05 959, this industrial bleaching process is enormously simplified with regard to the logistics regarding warehousing, transportation and mixing processes and is thus rendered more cost-effective in terms of operational costs. Moreover, the abrasives according to the invention have very good micro-biological stability, with uniform lightening/color for the lowest possible fat content being ensured over the entire bleaching campaign.

Compared to the methods known in the prior art, the degreasing proceeds more completely, meaning that effects and thus quality differences on the viscosity of the cosmetic end products can be ruled out.

The method of producing the abrasive substance according to the invention is illustrated in more detail with the help of Example 1.

### EXAMPLE 1

### Bleaching Process of Natural Shell or Kernel Flour with Hydrogen Peroxide

The bleaching process is carried out in a 1000 liter reaction container made of stainless steel with a high-speed stirrer and integrated double pH and temperature measurement.

The batch sizes are between 419.0 and 450.8 kg.

### Formulation for Example 1

| Raw material | Use amount/kg |
|---|---|
| Water | 215 to 220 |
| Shell or corn flour | 100 to 105 |
| particle size less than 200 μm | |
| Hydrogen peroxide 35% strength | 7.3 to 7.6 |
| Sodium hydroxide solution | 2.2 to 2.3 |
| 50% strength | |
| Water for washing | 135 to 150 |

The water is initially introduced in the reaction container and the abovementioned use amount of shell or corn flour is introduced with stirring. Two thirds of the bleaching agent amount of hydrogen peroxide solution are added to this suspension, and the sterilization process is carried out at a pH of 4 and 5.

The simultaneous metered addition of the remainder of the bleaching agent of hydrogen peroxide solution with 50% strength sodium hydroxide solution then takes place in the alkaline medium at a pH of from 7 to 10.

After 160 to 230 minutes, the bleaching process is concluded, where the temperature of the ready-bleached batch is about 15° C. above the starting temperature of the crude batch.

Water is removed from the resulting bleaching agent suspension, the bleached flour is continually washed, dried, containerized in large packs and passed directly to its further processing in cosmetic preparations.

Examples 2 and 4 give a solvent-free and a solvent-containing formulation according to the invention of a handwashing paste and of an anhydrous skin cleansing composition containing bleached shell and/or corn flour.

### EXAMPLE 2

### Formulation of a Solvent-free Handwashing Paste

| | Use amount/% by |
|---|---|
| Raw material | weight |
| Surfactant combination | 19.0 |
| consisting of sodium lauryl | |
| ether sulfate 26% strength and | |
| lauryl alcohol polyglycol ether | |
| Ricinus oil sulfonate 68% | 5.1 |
| strength | |
| Refined rapeseed oil | 9.0 |
| Water | 47.4 |
| Carboxymethylcellulose | 0.7 |
| Heteropolysaccharide, e.g. | 0.3 |
| xanthan gum | |
| Bleached shell or corn flour | 14.0 |
| Olein | 1.2 |
| Titanium dioxide | 0.5 |
| Citric acid | 0.3 |
| Sodium chloride | 1.5 |
| Preservative | 0.8 |
| Perfume | 0.2 |

### EXAMPLE 3

### Formulation of a Solvent-containing Handwashing Paste

| | Use amount/% by |
|---|---|
| Raw material | weight |
| Surfactant combination | 52.0 |
| consisting of sodium lauryl | |
| ether sulfate 28% strength and | |
| cocoamidopropylbetaine 30% | |
| strength | |
| Ricinus oil sulfonate 68% | 10.0 |
| strength | |
| Water | 0.8 |
| Carboxymethylcellulose | 0.5 |
| Organophilic bentonite | 2.1 |
| Tetra-n-butane (C₁₂-C₁₆-allkanes) | 17.0 |
| Commercial product from Oxeno | |
| Bleached olive kernel flour | 13.0 |
| Crystallized salt | 2.8 |
| Citric acid | 0.3 |
| Titanium dioxide | 0.5 |
| Preservative | 0.8 |
| Perfume | 0.2 |

### EXAMPLE 4

### Formulation of an Anhydrous Skin Cleansing Composition

| | Use amount/% by |
|---|---|
| Raw material | weight |
| Surfactant: fatty alcohol | 19.5 |
| C₁₂-C₁₈, 5EO | |
| Solvent consisting of dimethyl | 49.5 |
| adipate, glutarate, succinate | |
| Crosslinked polyacrylic acid | 4.0 |
| sodium salt | |
| Cellulose acetobutyrate | 3.5 |
| Isooctyl stearate | 3.9 |
| Bleached walnut shell flour | 13.0 |
| Organophilic bentonite | 4.4 |
| Propylene carbonate | 0.6 |
| Titanium dioxide | 1.0 |
| Fumed silica | 0.3 |
| Perfume | 0.3 |

The products are produced by the customary known methods which are generally known for the formulation of surfactant systems (G. Ziolkowski, Kosmetik-Jahrbuch [Cosmetics Annual] 1986, 1987, 1989, Verlag für Chemische Industrie, H. Ziolkowski K G, Augsburg, Kosmetik [Cosmetics] Georg-Thieme-Verlag Stuttgart).

The batches of bleached natural flours obtainable according to Example 1 were analyzed over a period of 24 months, during which 1100 samples were taken for the microbiological determination of the germ content and to establish the presence of pathogenic germs.

The results had far fewer contaminated samples from the tested big bags than was the case for the method according to DE 103 05 959. Whereas about 15% of the bleached material according to the method as in DE 103 05 959 had to be returned to the production process again on account of contamination, it is now merely about 0.2% by the method according to the invention. Moreover, the cost saving of the production process according to the invention compared with the previous process is about 30%.

Physical Color Measurement of the Degree of Lightness of the Process Products (L* value) Using a Calorimeter in Accordance with DIN 5033

To measure the degree of lightness, the measurement principle used was the color and color difference measurement by the 3-range method in accordance with DIN 5033.

The instrument used for this was the 3-range calorimeter MIKRO COLOR II from Dr Lange Bruno Lange GmbH Berlin Industriemeβtechnik, Dusseldorf with an optical construction as in DIN 5033. The light source used was a xenon flash lamp which, in conjunction with an Ulbricht sphere, serves for diffuse illumination of the sample to be measured—standard light type D65. According to DIN 5033, measurement is made here of the diffuse reflection of the sample under an angle of 8°. For the color measurement in accordance with DIN 5033, the reference standard or white standard used was the calibration standard LZM 076 certified in accordance with DIN 55350 part 18, 4.1.2:

| | |
|---|---|
| Standard number: | 010799 |
| Standard color value X: | 74.5 |
| Standard color value Y: | 79.5 |
| Standard color value Z: | 83.0 |
| Standard light type: | D65 |
| Normal observer: | 10° |
| Measurement geometry: | d/8° |

The table below gives the L* values of various batches of walnut shell flour bleached according to the invention.

Table of the color values:

| | | |
|---|---|---|
| Date | Batch number | Color L* value |

Method as in EP 0559 696 B1

| | | |
|---|---|---|
| Dec. 1, 1999 | 0040256813 | 70.5 |
| Dec. 2, 1999 | 0040256816 | 70.9 |
| Dec. 6, 1999 | 0040256817 | 71.4 |
| Dec. 16, 2000 | 0040290639 | 74.3 |
| Mar. 28, 2001 | 0040299877 | 72.3 |
| Sep. 11, 2002 | 0040344064 | 73.8 |
| Apr. 26, 2003 | 0040360168 | 74.7 |
| May 18, 2003 | 0040361975 | 71.9 |

Method according to the invention

| | | |
|---|---|---|
| Oct. 9, 2004 | 0040385914 | 81.8 |
| Oct. 17, 2004 | 0040396622 | 82.3 |

### Physical Measurement of the Fat Content

The tests for degreasing the flours obtained by the method according to the invention were carried out in accordance with the DGF unit method B-15 (87). This method is usually used for determining the oil content of oil seeds which are used as industrial starting materials for the production of fats and oils, and the method forms the basis for the evaluation of oil seeds according to their oil content.

Compared to the process products obtained according to DE 103 05 959, the flours obtained by the bleaching method according to the invention produced the following values in accordance with the aforementioned DGF unit method:

Table of the fat contents:

| | | |
|---|---|---|
| Date | Batch number | Fat content/% |

Method as in EP 0559 696 B1
or DE 103 05 959

| | | |
|---|---|---|
| Jun. 29, 2000 | 0040264186 | 0.08 |
| Jul. 1, 2001 | 0040306700 | 0.12 |
| Feb. 23, 2002 | 0040326688 | 0.08 |
| May 18, 2003 | 0040361975 | 0.10 |

Method according to the invention

| | | |
|---|---|---|
| Dec. 17, 2003 | 0040376537 | 0.05 |
| Sep. 3, 2004 | 0040385945 | 0.05 |
| Oct. 3, 2004 | 0040385908 | 0.05 |
| Oct. 14, 2004 | 0040396619 | 0.04 |
| Oct. 17, 2004 | 0040396622 | 0.05 |

As a result of the method according to the invention, the bleached flours have a significantly lower fat content, meaning that effects on the quality of the cosmetic preparations which comprise these flours as abrasives can be ruled out.

Compared with the methods as in EP 559 696 B1 and DE 103 05 959, the degreasing proceeded more completely, meaning that effects and thus quality differences on the viscosity of the end products were largely ruled out.

1: A method of producing an abrasive substance, where natural kernels, shells, fruit skins and/or seeds are ground to give a flour of defined particle size, the flour is treated in aqueous suspension with at least one bleaching agent, wherein the addition of the bleaching agent takes place in two steps, where, in the first step, at least 50% by weight to 90% by weight of the total amount of bleaching agent are added and the flour is sterilized in the acidic medium and, in the second step, the lightening and degreasing of the flour is brought about by the simultaneous metered addition of the remainder of the bleaching agent with an alkali solution in alkaline medium.

2: The method as claimed in claim 1, wherein at least one bleaching agent is added to the bleach material in an amount of from 1.0 to 10.0% by weight, based on the total batch amount.

3: The method as claimed in claim 1, wherein aqueous hydrogen peroxide solution is used as bleaching agent.

4: The method as claimed in claim 1, wherein the sterilization takes place in the acidic medium at a pH of from 3 to 5.

5: The method as claimed in claim 1, wherein the lightening and degreasing of the flour is brought about at a pH of from 7 to 11.

6: The method as claimed in claim 1, wherein the remainder of the bleaching agent is 50 to 10% by weight.

7: The method as claimed in claim 1, wherein the natural kernel, shell, fruit skin and/or seed flours used are walnut, hazelnut, almond shell flour, olive, apricot, peach, cherry or plum kernel flour, flours of palm kernels and coconut, jojoba fruits, macadamia nuts, pistachios and pine shells, corn cob flour, wheat bran, oat flour or wood flours, and any desired mixtures thereof with a particle size of from 50 to 2000 μm.

8: The method as claimed in claim 1, wherein the end temperature of the natural kernel, shell, fruit skin and/or seed flour suspension after the bleaching agent treatment is essentially 15 to 20° C. above the starting temperature of the bleaching treatment.

9. A method for cleaning skin comprising applying an anhydrous or hydrous skin cleansing composition to the skin, which comprises the abrasive substance as claimed in claim 1 and surfactants, soaps or other emulsifiers, organic solvents or oils, and optionally thickeners, builders, dyes and antioxidants, further comprising a content of bleached, finely divided, natural kernel, shell, fruit skin and/or seed flour as abrasive for aiding skin cleansing.
